(19) United States
(12) Patent Application Publication
(10) Pub. No.: US 2023/0100114 A1
(43) Pub. Date: Mar. 30, 2023

(54) PD-CATALYZED AMINATION OF FLUORINATED ARYL CHLORIDES

(71) Applicant: BASF SE, Ludwigshafen (DE)

(72) Inventors: Kailaskumar Borate, Navi Mumbai (IN); Saravanan Thirunavukkarasu, Navi Mumbai (IN); Rahul Kaduskar, Navi Mumbai (IN); Harish Shinde, Navi Mumbai (IN); Roland Goetz, Ludwigshafen (DE)

(21) Appl. No.: 17/917,301

(22) PCT Filed: Apr. 6, 2021
(86) PCT No.: PCT/EP2021/058892

§ 371 Date: Oct. 6, 2022

(30) Foreign Application Priority Data
Apr. 10, 2020 (EP) 20169216.7

(51) Int. Cl. C07C 209/10; B01J 31/22; B01J 31/18

(52) U.S. Cl. CPC C07C 209/10; B01J 31/2226; B01J 31/189; B01J 2531/824; B01J 2231/44

(57) ABSTRACT

The presently claimed invention relates to a process for the preparation of di-, tri-, or tetra fluoroarylamine by reacting polyfluorinated aryl chlorides with ammonia in the presence of a base, a metal catalyst and a ligand. Di-, tri-, tetrafluoroarylamines are valuable intermediates and find application in several areas, mainly in epoxy polymers, colorants, dyes, polyurethanes, agrochemicals and pharmaceutical active agents.

### FIELD OF THE INVENTION

The presently claimed invention relates to a process for the preparation of di-, tri-, or tetra-fluoroarylamine.

### BACKGROUND OF THE INVENTION

Di-, tri-, tetrafluoroarylamines are valuable intermediates and find application in several areas, mainly in epoxy polymers, colorants, dyes, polyurethanes, agrochemicals and pharmaceutical active agents.

Organic Letters 2014, 16, 4388-4391, discloses a palladium-catalyzed amination of aryl chlorides and bromides with ammonium salts and a palladium-catalyzed coupling of aryl halides with ammonia and gaseous amines.

WO 2004/054961 A1 discloses a process for the preparation of substituted halogenated aniline by reacting halogenated 1-chlorobenzene with an imine in the presence of a transition metal catalyst complex and a base and further hydrolyzing the N-aryl imide.

Polyfluorinated amines are generally synthesised via the nitration of polyfluorinated aryls followed by reduction. Nitration of polyfluorinated aromatic compounds is a critical step in the synthesis, requires a high energy input and also generates a high effluent load.

Hence, there is a need to devise alternative synthetic processes for the preparation of polyfluorinated amines.

However, the processes of the prior art suffer from several drawbacks, such as a high effluent load and a high metal salt load, low yields, a low selectivity and the requirement of using non-benign solvents.

Hence, it is an object of the presently claimed invention to provide an economical process for the preparation of di-, tri-, or tetrafluoroarylamines in a high overall yield, i.e. ≥60% or ≥80%, with high selectivity, that can be easily controlled, i.e. does not become exothermic.

### SUMMARY OF THE INVENTION

Surprisingly, it has been found that di-, tri-, or tetra fluoroarylamines are obtained in a high overall yield, i.e. ≥60% or ≥80%, with high selectivity by reacting polyfluorinated aryl chlorides with ammonia in the presence of a base, a metal catalyst and a ligand.

Accordingly, in one aspect, the presently claimed invention is directed to a process for the preparation of a di-, tri-, or tetra fluoroarylamine comprising at least the step of:

a) amination of a di-, tri-, or tetra fluoroarylchloride in the presence of
i. ammonia,
ii. at least one base,
iii. at least one metal catalyst complex,
iv. at least one ligand, and
v. at least one organic solvent;

wherein the at least one catalyst and the at least one ligand are present in a molar ratio in the range of 1:1 to 1:10.

### DETAILED DESCRIPTION

Before the present compositions and formulations of the presently claimed invention are described, it is to be understood that this invention is not limited to particular compositions and formulations described, since such compositions and formulation may, of course, vary. It is also to be understood that the terminology used herein is not intended to be limiting, since the scope of the presently claimed invention will be limited only by the appended claims.

If hereinafter a group is defined to comprise at least a certain number of embodiments, this is meant to also encompass a group which preferably consists of these embodiments only. Furthermore, the terms ‘first’, ‘second’, ‘third’ or ‘a’, ‘b’, ‘c’, etc. and the like in the description and in the claims, are used for distinguishing between similar elements and not necessarily for describing a sequential or chronological order. It is to be understood that the terms so used are interchangeable under appropriate circumstances and that the embodiments of the presently claimed invention described herein are capable of operation in other sequences than described or illustrated herein. In case the terms ‘first’, ‘second’, ‘third’ or ‘(A)’, ‘(B)’ and ‘(C)’ or ‘(a)’, ‘(b)’, ‘(c)’, ‘(d)’, ‘ii’ etc. relate to steps of a method or use or assay there is no time or time interval coherence between the steps, that is, the steps may be carried out simultaneously or there may be time intervals of seconds, min, hours, days, weeks, months or even years between such steps, unless otherwise indicated in the application as set forth herein above or below.

Furthermore, the ranges defined throughout the specification include the end values as well, i.e. a range of 1 to 10 implies that both 1 and 10 are included in the range. For the avoidance of doubt, applicant shall be entitled to any equivalents according to applicable law.

In the following passages, different aspects of the presently claimed invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to ‘one embodiment’ or ‘an embodiment’ means that a particular feature, structure or characteristic described in connection with the embodiment is included in at least one embodiment of the presently claimed invention. Thus, appearances of the phrases ‘in one embodiment’ or ‘in an embodiment’ in various places throughout this specification are not necessarily all referring to the same embodiment, but may refer to the same embodiment. Further, as used in the following, the terms “preferably”, “more preferably”, “even more preferably”, “most preferably” and “in particular” or similar terms are used in conjunction with optional features, without restricting alternative possibilities. Thus, features introduced by these terms are optional features and are not intended to restrict the scope of the claims in any way.

Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the presently claimed invention, and form different embodiments, as would be understood by those in the art. For example, in the appended claims, any one of the claimed embodiments can be used in any combination.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

The presently claimed invention relates to a process for the preparation of a di-, tri-, or tetra-fluoroarylamine comprising at least the step of:

a) amination of a di-, tri-, or tetra fluoroarylchloride in the presence of
i. ammonia,
ii. at least one base,
iii. at least one metal catalyst complex,
iv. at least one ligand, and
v. at least one solvent;

wherein the at least one catalyst and the at least one ligand are present in a molar ratio in the range of 1:1 to 1:10.

The process of the instant invention can be represented by the generalized reaction depicted in Scheme I.

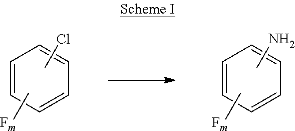

In an embodiment of the presently claimed invention, the di-, tri-, or tetra fluoroarylamine is selected from the group consisting of compounds of general formula (A),

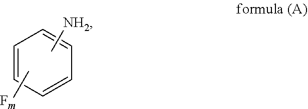

wherein m is 2, 3 or 4.

In an embodiment of the presently claimed invention, the compound of formula (A) is a difluoroarylamine.

In yet another embodiment of the presently claimed invention, the compound of formula (A) is a trifluoroarylamine.

In a further embodiment of the presently claimed invention, the compound of formula (A) is a tetrafluoroarylamine.

In an embodiment of the presently claimed invention, the di-, tri-, or tetra fluoroarylchloride is selected from the group consisting of compounds of general formula (B),

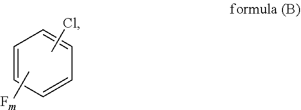

wherein m is 2, 3 or 4.

In an embodiment of the presently claimed invention, the di-, tri- or tetra fluoroarylchloride is selected from the group consisting of 1,2-fluorochlorobenzene; 1,3-fluorochlorobenzene; 3,4-difluorochlorobenzene; 2,5-difluorochlorobenzene; 2,3-difluorochlorobenzene; 3,4-trifluorochlorobenzene, 2,3,4-Trifluoro chlorobenzene and 2,3,5,6-tetra-fluorochlorobenzene.

In an embodiment of the presently claimed invention, ammonia can used in the form of a solution in at least one organic solvent or in gaseous form.

If the ammonia is dissolved in at least one organic solvent before mixing with the other components, i.e. the starting material, the at least one base, the at least one catalyst complex, the at least one ligand and the at least one organic solvent, then the at least organic solvent for dissolving the ammonia is preferably identical to the at least one organic solvent for conducting the reaction.

In yet another embodiment of the presently claimed invention, the at least one organic solvent is selected from the group consisting of cyclic ethers, acyclic aliphatic ethers and aliphatic alcohols.

In a further embodiment of the presently claimed invention, the at least one organic solvent is selected from the group consisting of 1,4-dioxane, 1,3-dioxane, tetrahydrofuran, cyclopentyl methyl ether, tert-butyl-methyl-ether, methanol and isopropanol.

In a preferred embodiment of the presently claimed invention, the at least one solvent is 1,4-dioxane or 1,3-dioxane.

In an embodiment, the at least one base is selected from the group consisting of alkoxides, carbonates, bicarbonates, hydroxides, amides, amines, phosphates and fluorides.

In an embodiment, the at least one base is selected from the group consisting of alkoxides, such as sodium tert-butoxide, potassium tert-butoxide and sodium methoxide.

In an embodiment of the presently claimed invention, the at least one base is selected from the group consisting of alkali metal amides, such as sodium amide and lithium diisopropylamide, alkali metal bis(trialkylsilyl)amides such as lithium bis-(trimethyl-silyl)amide and sodium bis-(trimethyl-silyl)amide, amines such as triethylamine, tributylamine, trimethylamine, diisopropyl ethyl amine, pyridine, N,N-dimethylaminopyridine, 1,5-diazabicycl[4.3.0]nonene-5,1,4-diazabicyclo[2.2. 2]octane and 1,5-diazabicycl-[5.4.0]undecene-5.

In an embodiment of the presently claimed invention, the at least one base is selected from the group consisting of an alkali, alkaline earth carbonate, bicarbonate, hydroxide, phosphates and fluorides, such as sodium carbonate, sodium bicarbonate, sodium hydroxide, magnesium carbonate, magnesium bicarbonate, magnesium hydroxide, calcium carbonate, calcium bicarbonate, calcium hydroxide, barium carbonate, barium hydroxide, barium bicarbonate, potassium carbonate, potassium bicarbonate, potassium hydroxide, sodium acetate, potassium acetate, potassium phosphate, calcium acetate, caesium fluoride, potassium hydrogen phosphate, sodium phosphate, sodium hydrogen phosphate, sodium dihydrogen phosphate, potassium dihydrogen phosphate sodium trifluoroacetate, potassium trifluoroacetate, caesium carbonate, caesium bicarbonate and caesium hydroxide.

In a preferred embodiment of the presently claimed invention, the at least one base is sodium tert-butoxide or potassium tert-butoxide.

In an embodiment, the at least one metal catalyst complex is selected from the group consisting of transition metal catalysts including soluble or insoluble complexes of platinum, palladium and nickel. Nickel and palladium are particularly preferred, and palladium is most preferred.

In an embodiment, the at least one metal catalyst complex is selected from the group consisting of tetrakis(triphenylphosphine) palladium, dichlorobis(triphenylphosphine) palladium, tris(dibenzylideneacetone) dipalladium [Pd₂(dba)₃], bis(dibenzylideneacetone) dipalladium [Pd(dba)₂], palladium acetate, dichloro(1,5-cyclooctadiene) palladium and bis[cinnamyl palladium(II)] chloride.

In a preferred embodiment, the at least one metal catalyst complex is bis[cinnamyl palladium(II)] chloride.

In an embodiment, the at least one ligand is selected from the group consisting of 5-(di-tert-butylphosphino)-1′,3′,5′-triphenyl-1′H-1,4′-bipyrazole, bis(2-methyl-2-propanyl)(2′,4′,6′-triisopropyl-3,6-dimethoxy-2-biphenylyl)phosphine, dicyclohexyl(2′,4′,6′-triisopropyl-3,6-dimethoxy-[1,1′-biphenyl]-2-yl)phosphine, bis(2-methyl-2-propanyl)(2′,4′,6′-triisopropyl-2-biphenylyl)phosphine, di-(1-adamantyl)-2-morpholinophenylphosphine, tributylphosphine, butyl-di-1-adamantyl phosphine, (5-diphenylphosphanyl-9,9-dimethylxanthen-4-yl)-diphenylphosphane, (R)-1-[(SP)-2-(diphenylphosphino)ferrocenyl]ethyldicyclohexylphosphine, dicyclohexyl-[2-[2,6-di(propan-2-yloxy)phenyl]phenyl]phosphane, bis[5-(di(1-adamantyl)phosphino)-1′,3′,5′-triphenyl-1′H-[1,4′]bipyrazole, trimethylphosphine, tri-ethylphosphine, tripropylphosphine, triisopropylphosphine, tributylphosphine, tricyclohexylphosphine, trimethylphosphine, triethylphosphite, tripropylphosphite, triisopropylphosphite, tributylphosphite, tricyclohexylphosphite, triphenylphosphine, tri(o-tolyl)phosphine, triisopropylphosphine, tricyclohexylphosphine, 2,2′-bis(diphenylphosphino)-1,1′-binaphthyl (BINAP), 1,2-bis(dimethylphosphino)ethane, 1,2-bis(diethylphosphino)-ethane, 1,2-bis(dipropylphosphino)ethane, 4,5-bis(diphenylphosphino)-9,9-dimethyl-xanthene (xantphos), 1,1′-bis(diphenylphosphino)ferrocene (dppf), bis(2-(diphenyl-phosphino)phenyl)ether [DPE-phos], 1,2-bis(diisopropylphosphino)ethane, 1,2-bis-(dibutylphosphino)ethane, 1,2-bis(dicyclohexylphosphino)ethane, 1,3-bis(diisopropyl-phosphino)propane, 1,3-bis(dicyclohexylphosphino)propane, 1,4-bis(diisopropyl-phosphino)butane, 1,4-bis(dicyclohexylphosphino)butane, 1,4-bis(diphenylphosphino)-butane (bppb), 2,4-bis(dicyclohexylphosphino)pentane and 1,1′-bis(diphenylphosphino)ferrocene (dppf).

In yet another embodiment of the presently claimed invention, the at least one ligand is selected from the group consisting of 5-(di-tert-butylphosphino)-1′,3′,5′-triphenyl-1′H-1,4′-bipyrazole, and bis[5-(di(1-adamantyl)phosphino)-1′,3′,5′-triphenyl-1′H-[1,4′]bipyrazole.

In yet another embodiment of the presently claimed invention, the at least one metal catalyst complex and the at least one ligand are present in a molar ratio in the range of 1:2 to 1:6.

In a further embodiment of the presently claimed invention, the at least one metal catalyst complex and the at least one ligand are present in a molar ratio of 1:2, 1:3, 1:4, 1:5, or 1:6.

In a preferred embodiment of the presently claimed invention, the at least one metal catalyst complex and the at least one ligand are present in a molar ratio of 1:4.

In a preferred embodiment of the presently claimed invention, the reaction is carried out a temperature in the range of ≥80° C. to ≥140° C., and more preferably a temperature in the range of ≥90° C. to ≤120° C.

In an embodiment of the presently claimed invention, the reaction is carried out at a pressure in the range of bar to 30 bar, and more preferably at a pressure in the range of 1 to 10 bar.

In an embodiment of the presently claimed invention, the amination is carried out in the presence of ammonia, sodium tert-butoxide or potassium tert-butoxide, bis[cinnamyl palladium(II)] chloride, 5-(di-tert-butylphosphino)-1′,3′,5′-triphenyl-1′H-1,4′-bipyrazole and 1,4-dioxane.

In an embodiment of the presently claimed invention, the amination is carried out in the presence of ammonia, sodium tert-butoxide, bis[cinnamyl palladium(II)] chloride, 5-(di-tert-butylphosphino)-1′,3′,5′-triphenyl-1′H-1,4′-bipyrazole or bis[5-(di(1-adamantyl)phosphino)-1′,3′,5′-triphenyl-1′H-[1,4′]bipyrazole and 1,4-dioxane.

In an embodiment of the presently claimed invention, the amination of a difluoroarylchloride is carried out in the presence of ammonia, sodium tert-butoxide, bis[cinnamyl palladium(II)] chloride, 5-(di-tert-butylphosphino)-1′,3′,5′-triphenyl-1′H-1,4′-bipyrazole or bis[5-(di(1-adamantyl)phosphino)-1′,3′,5′-triphenyl-1′H-[1,4′]bipyrazole and 1,4-dioxane to yield difluoroarylamine.

In an embodiment of the presently claimed invention, the amination of a trifluoroarylchloride is carried out in the presence of ammonia, sodium tert-butoxide, bis[cinnamyl palladium(II)] chloride, 5-(di-tert-butylphosphino)-1′,3′,5′-triphenyl-1′H-1,4′-bipyrazole or bis[5-(di(1-adamantyl)phosphino)-1′,3′,5′-triphenyl-1′H-[1,4′]bipyrazole and 1,4-dioxane to yield trifluoroarylamine.

Advantages

The presently claimed invention is associated with at least one of the following advantages:

(i) The reaction sequence is carried out in one pot.

(ii) Substituted aryl amines are obtained in high yields.

(iii) Similar reaction conditions can be applied to obtain products with di, tri and tetra substitution of fluorine.

(iv) High pressure increases the selectivity of the product.

(v) The process of the presently claimed invention is safe and can be easily controlled.

(vi) The process of the presently claimed invention is economical.

In the following, there is provided a list of embodiments to further illustrate the present disclosure without intending to limit the disclosure to the specific embodiments listed below.

### Embodiments

1. A process for the preparation of a di-, tri-, or tetrafluoroarylamine comprising at least the step of:
a) amination of a di-, tri-, or tetra fluoroarylchloride in the presence of
i) ammonia,
ii) at least one base,
iii) at least one metal catalyst complex,
iv) at least one ligand, and
v) at least one organic solvent;
wherein the at least one catalyst and the at least one ligand are present in a molar ratio in the range of 1:1 to 1:10.

2. The process according to embodiment 1, wherein the at least one catalyst and the at least one ligand are present in a molar ratio in the range of 1:2 to 1:6.

3. The process according to embodiment 1 or 2, wherein the di-, tri-, or tetrafluoroarylamine is selected from the group consisting of compounds of formula (A)

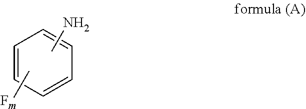

wherein m is 2, 3 or 4.

4. The process according to any one of the embodiments 1 to 3, wherein the di-, tri-, or tetra fluoroarylchloride is selected from the group consisting of compounds of formula (B)

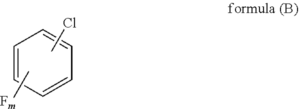

wherein m is 2, 3 or 4.

5. The process according to any one of the embodiments 1 to 4, wherein the at least one organic solvent is selected from the group consisting of cyclic ethers, acyclic aliphatic ethers and aliphatic alcohols.

6. The process according to any one of the embodiments 1 to 5, wherein the at least one organic solvent is selected from the group consisting of 1,4-dioxane, 1,3-dioxane, tetrahydrofuran, cyclopentyl methyl ether, tert-butyl-methyl-ether, methanol and isopropanol.

7. The process according to any one of the embodiments 1 to 6, wherein the at least one base is selected from the group consisting of alkoxides, carbonates, bicarbonates, hydroxides, amides, amines, phosphates and fluorides.

8. The process according to any one of the embodiments 1 to 7, wherein the at least one base is selected from the group consisting of sodium tert-butoxide, potassium tert-butoxide, sodium methoxide, sodium amide, lithium diisopropylamide, lithium bis-(trimethyl-silyl)amide, sodium bis-(trimethyl-silyl)amide, triethylamine, trimethylamine, N-dimethylaminopyridine, 1,5-diazabicycl[4.3.0]nonene-5, 5-diazabicycl [5.4.0]undecene-5, lutidine, sodium carbonate, sodium bicarbonate, sodium hydroxide, magnesium carbonate, magnesium bicarbonate, magnesium hydroxide, calcium carbonate, calcium bicarbonate, calcium hydroxide, barium carbonate, barium hydroxide, barium bicarbonate, potassium carbonate, potassium bicarbonate, potassium hydroxide, sodium acetate, potassium acetate, potassium phosphate, calcium acetate, caesium fluoride, potassium hydrogen phosphate, sodium phosphate, sodium hydrogen phosphate, sodium dihydrogen phosphate, potassium dihydrogen phosphate, tributyl amine, pyridine, 1,4-diazabicyclo[2.2. 2]octane, tetramethylguanidine, diisopropyl ethyl amine, sodium trifluoroacetate, potassium trifluoroacetate caesium carbonate, caesium bicarbonate and caesium hydroxide.

9. The process according to any one of the embodiments 1 to 8, wherein the at least one base is sodium tert-butoxide.

10. The process according to any one of the embodiments 1 to 9, wherein the at least one metal catalyst complex comprises at least one metal and at least one complexing agent.

11. The process according to embodiment 10, wherein the at least one metal is selected from the group consisting of nickel, palladium and platinum.

12. The process according to any one of the embodiments 1 to 11, wherein the at least one metal catalyst complex is selected from the group consisting of tetrakis(triphenylphosphine) palladium, dichlorobis(triphenylphosphine) palladium, tris(dibenzylideneacetone) dipalladium [Pd₂(dba)₃], bis(dibenzylideneacetone) dipalladium

[Pd(dba)₂], palladium acetate, dichloro(1,5-cyclooctadiene) palladium and bis[cinnamyl palladium(II)] chloride.

13. The process according to any one of the embodiments 1 to 12, wherein the at least one metal catalyst complex is bis[cinnamyl palladium(II)] chloride.

14. The process according to any one of the embodiments 1 to 13, wherein the at least one ligand is selected from the group consisting of 5-(di-tert-butylphosphino)-1′,3′,5′-triphenyl-1′H-[1,4′]-bipyrazole, bis(2-methyl-2-propanyl)(2′,4′,6′-triisopropyl-3,6-dimethoxy biphenylyl)phosphine, dicyclohexyl(2′,4′,6′-triisopropyl-3,6-dimethoxy-[1,1′-biphenyl] yl)phosphine, bis(2-methyl-2-propanyl)(2′,4′,6′-triisopropyl-2-biphenylyl)phosphine, di-(1-adamantyl)-2-morpholinophenylphosphine, tributylphosphine, butyldiadamantylphosphine, (5-diphenylphosphanyl-9,9-dimethylxanthen-4-yl)-diphenylphosphane, (R)-1-[(SP) (diphenylphosphino)ferrocenyl]ethyldicyclohexylphosphine, dicyclohexyl-[2-[2,6-di(propan-2-yloxy)phenyl]phenyl]phosphane, bis[5-(di(1-adamantyl)phosphino)-1′,3′,5′-triphenyl-1′H-[1,4′]bipyrazole, trimethylphosphine, triethylphosphine, tripropylphosphine, triisopropylphosphine, tributylphosphine, tricyclohexylphosphine, trimethylphosphine, triethyl phosphite, tripropylphosphite, triisopropylphosphite, tributylphosphite, tricyclohexylphosphite, triphenylphosphine, tri(o-tolyl)phosphine, triisopropylphosphine, tricyclohexylphosphine, 2,2′-bis(diphenylphosphino)-1,1′-binaphthyl (BINAP), 1,2-bis(dimethylphosphino)ethane, 1,2-bis(diethylphosphino)-ethane, 1,2-bis(dipropylphosphino)ethane, 4,5-bis(diphenylphosphino)-9,9-dimethyl-xanthene (xantphos), 1,1′-bis(diphenylphosphino)ferrocene (dppf), bis(2-(diphenyl-phosphino)phenyl)ether [DPE-phos], 1,2-bis(diisopropylphosphino)ethane, 1,2-bis-(dibutylphosphino)ethane, 1,2-bis(dicyclohexylphosphino)ethane, 1,3-bis(diisopropyl-phosphino) propane, 1,3-bis(dicyclohexylphosphino)propane, 1,4-bis(diisopropyl-phosphino)butane, 1,4-bis(dicyclohexylphosphino)butane, 1,4-bis(diphenylphosphino)-butane (bppb), 2,4-bis(dicyclohexylphosphino)pentane and 1,1′-bis(diphenylphosphino)ferrocene (dppf).

15. The process according to any one of the embodiments 1 to 14, wherein the at least one ligand is 5-(di-tert-butylphosphino)-1′,3′,5′-triphenyl-1′H-1,4′-bipyrazole or bis[5-(di(1-adamantyl) phosphino)-1′,3′,5′-triphenyl-1′H-[1,4′]bipyrazole.

16. The process according to any one of the embodiments 1 to 15, wherein the at least one base is selected from the group consisting of sodium tert-butoxide and potassium tert-butoxide,
the at least one metal complex is bis[cinnamyl palladium(II)] chloride,
the at least one ligand is selected from the group consisting of 5-(di-tert-butylphosphino)-1′,3′,5′-triphenyl-1′H-1,4′-bipyrazole and bis[5-(di(1-adamantyl)phosphino)-1′,3′,5′-triphenyl-1′H-[1,4′]bipyrazole, and
the at least one organic solvent is selected from the group consisting of 1,4-dioxane, 1,3-dioxane, tetrahydrofuran, cyclopentyl methyl ether, tert-butyl-methyl-ether, methanol and isopropanol.

17. The process according to any one of the embodiments 1 to 16, wherein step a) is carried out at a temperature in the range of 80 ° C. to 140 ° C.

18. The process according to any one of the embodiments 1 to 17, wherein step a) carried out at a temperature in the range of 90 ° C. to 120 ° C.

19. The process according to any one of the embodiments 1 to 18, wherein step a) is carried out at a pressure in the range of bar to 30 bar.

20. The process according to any one of the embodiments 1 to 19, wherein step a) is carried out at a pressure of 1 to 10 bar.

### Examples

The presently claimed invention is illustrated in detail by non-restrictive working examples which follow.

Materials

2,3,4-trifluorobenzene (2,3,4-TFCB) was obtained from Fluorochem

DBU (5-diazabicycl [5.4.0]undecene-5) was obtained from Spectrochem

NaTFA (Sodium trifluro acetic acid)was obtained from Spectrochem

Na-tert-butoxide was obtained from Spectrochem

Na methoxide was obtained from Spectrochem

Bippyphos (5-(di-tert-butylphosphino)-1′,3′,5′-triphenyl-1′H-1,4′-bipyrazole)was obtained from Sigma Aldrich

JosiPhos ((R)-1-[(SP)-2-(diphenylphosphino)ferrocenyl]ethyldicyclohexylphosphine) was obtained from Labnetwork

Bis[cinnamyl palladium(II) chloride] was obtained from Sigma Aldrich

[Pd(o-tol)₃P]₂ was obtained from Labnetwork

Methods

The characterization was by coupled gas Chromatography/mass spectrometry (GC/MS),

GCMS method 1:

Instrument: Shimadzu [MS-QP2020NX], Shimadzu [GC-QP2010 Ultra]

System: GC-MS

GC Method Details:

Mobile Phase: Inert gas (Helium); Column Oven Temp. : 50.0° C; Injection Temp.: 250.00° C.; Injection Mode: Split; Flow Control Mode: Linear Velocity; Pressure: 120.0 kPa; Total Flow: 15.3 mL/min; Column Flow: 0.40 mL/min; Linear Velocity: 28.0 cm/sec; Purge Flow: 3.0 mL/min

Split Ratio: 30.0; Run Time: 8.0 min; Injection volume: 0.20-1.0 [symbol] L; Column: SH-Rxi-5 Sil MS (20 meter, 0.15 mm ID, 0.15 um).

Sample Preparation: In Methanol and Acetonitrile (Approx. 2 mg sample in 1.0 mL Methanol or Acetonitrile)

Oven temperature program: 50° C., hold time 1 min; rate of increase 50° C./min to 300 ° C. and hold time 2 min at 300° C.

MS Method Details:

Ionization source: Electron Impact Ionisation; Ion Source Temp: 230.00° C.; Interface Temp.: 280.00° C.; Start m/z: 50.00; End m/z: 800.00.

Abbreviations used are: h for hour(s), min for minute(s), rt for retention time and ambient temperature for 20-25° C.

### Example 1

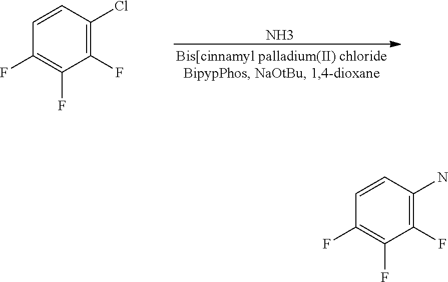

In a 10 ml Mya-4 glass vial 2,3,4-trifluorobenzene (0.1 g, 1 eq), NaOtBu (80 mg, 1.5 eq), Bis[cinnamyl palladium(II) chloride] (3.1 mg, 1 mole %), Bippyphos (12 mg, 4 mole %) were mixed and then ammonia solution (0.4 M) in 1,4-dioxane (4 ml, 3 eq) was added. Then a magnetic stir bar was added, and the vial was sealed and placed in aluminum block and heated at 100° C. for 18 hrs (overnight).

The reaction mass was analyzed on GCMS after filtering though syringe filter (0.5 μ).

### Example 2

Reaction in Autoclave

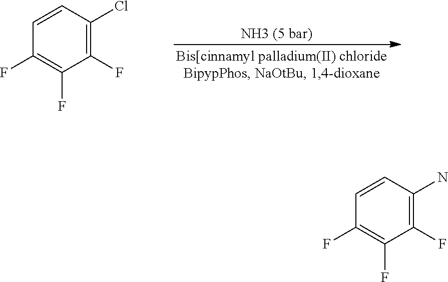 [SMILES: Fc1ccc(Cl)c(F)c1F ; "NH3 (5 bar)" ; Nc1ccc(F)c(F)c1F]

In 100 ml autoclave vessel, 2,3,4-trifluorobenzene (1000 mg), NaOtBu(860 mg, 1.5 eq), Bis[cinnamyl palladium(II) chloride] (31 mg, 1 mole %), Bippyphos (121 mg, 4 mole%) followed by 30 ml 1,4-dioxane and vessel was sealed, inertised and pressurized with 5 bar NH₃ pressure and heated to 100° C. for 18 hrs (overnight).

The reaction mass was analyzed on GCMS after filtering though syringe filter (0.5 μ).

Work Up

The reaction mixture was filtered through celite bed and the bed was washed with 1,4-dioxane (10 ml). The combined 1,4-dioxane solution was twice diluted with 30 ml dichloromethane and washed with water (100 ml). The obtained dichloromethane layer was concentrated to dryness.

The following table 1 gives an overview of different examples 3 to 12 which were carried out in accordance with example 1 by varying ammonia sources, bases and catalyst/ligands.

TABLE 1

| | | | | | Product | |
|---|---|---|---|---|---|---|
| | | | | Catalyst/ligand | ArNH₂ % | Ar₂NH % |
| Ex. | Starting material (1 eq.) | Ammonia source | Base | system | conversion | conversion |
| 1 | 2,3,4- | 0.5M NH₃ in 1,4- | Na-tert-butoxide | Bis[cinnamyl palladium(II) chloride] | 62 | 31 |
| | Trifluorochlorobenzene | dioxane (3 eq) | 1.4 eq | (1 mole %); Bippyphos (4 mole %) | | |
| 2 | 2,3,4- | gaseous | Na-tert-butoxide | Bis[cinnamyl palladium(II) chloride] | 80 | 17 |
| | Trifluorochlorobenzene | ammonia 5 bar | 1.5 eq | (1 mole %); Bippyphos (4 mole %) | | |
| 3 | 2,3,4- | gaseous | Na-tert-butoxide | Bis[cinnamyl palladium(II) chloride] | 82 | 4 |
| | Trifluorochlorobenzene | ammonia 7 bar | 1.5 eq | (1 mole %); Bippyphos (4 mole %) | | |
| 4 | 2,5- | gaseous | Na-tert-butoxide | Bis[cinnamyl palladium(II) chloride] | 99 | 1 |
| | difluorochlorobenzene | ammonia 7 bar | 1.4 eq | (1 mole %); Bippyphos (4 mole %) | | |
| 5 | 2,3,5,6-tetrafluoro | gaseous | Na-tert-butoxide | Bis[cinnamyl palladium(II) chloride] | 42 | 0 |
| | chlorobenzene | ammonia 7 bar | 1.4 eq | (1 mole %); Bippyphos (4 mole %) | | |
| 6* | 2,3,4-trifluorochlorbezene | aqueous | Na methoxide | JosiPhos/[Pd(o-tol)₃P]₂ | 0 | 0 |
| | | ammonia | | | | |
| 7* | 2,3,4-trifluorochlorbezene | (NH₄)₂SO₄ 1.5 eq | Na methoxide | JosiPhos/[Pd(o-tol)₃P]₂ | 0 | 0 |
| 8* | 2,3,4-trifluorochlorbezene | NH₄Cl 3.0 eq | Na methoxide | JosiPhos/[Pd(o-tol)₃P]₂ | 0 | 0 |
| 9* | 2,3,4-trifluorochlorbezene | (NH₄)₂SO₄ 1.5 eq | Na-tert-butoxide | JosiPhos/[Pd(o-tol)₃P]₂ | 2 | 0 |
| 10* | 2,3,4-trifluorochlorbezene | (NH₄)₂SO₄ 1.5 eq | DBU/NATFA | JosiPhos/[Pd(o-tol)₃P]₂ | 0 | 0 |
| 11* | 2,3,4-trifluorochlorbezene | gaseous | Na-tert-butoxide | Bis[cinnamyl palladium(II) chloride] | 0 | 0 |
| | | ammonia 7 bar | 1.5 eq | (1 mole %); DPPB (4 mole %) | | |
| 12* | 2,3,5,6- tetrafluoro | gaseous | Na-tert-butoxide | Bis[cinnamyl palladium(II) chloride] | 0 | 0 |
| | chlorobenzene | ammonia 7 bar | 1.5 eq | (1mole %); DPPF(4 mole %) | | |

*Not as per the invention

1. A process for the preparation of a di-, tri-, or tetra fluoroarylamine comprising at least the step of:
a) amination of a di-, tri-, or tetra fluoroarylchloride in the presence of
i) ammonia,
ii) at least one base,
iii) at least one metal catalyst complex
iv) at least one ligand, and
v) at least one organic solvent;
wherein the at least one catalyst and the at least one ligand are present in a molar ratio in the range of 1:1 to 1:10.

2. The process according to claim 1, wherein the di-, tri-, or tetra fluoroarylamine is selected from the group consisting of compounds of formula (A), 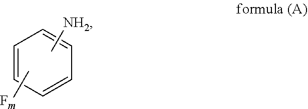
wherein m is 2, 3 or 4.

3. The process according to claim 1, wherein the di-, tri-, or tetra fluoroarylchloride is selected from the group consisting of compounds of formula (B), 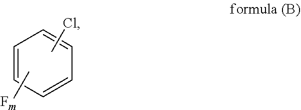
wherein m is 2, 3 or 4.

4. The process according to claim 1, wherein the at least one organic solvent is selected from the group consisting of cyclic ethers, acyclic aliphatic ethers, and aliphatic alcohols.

5. The process according to claim 1, wherein the at least one organic solvent is selected from the group consisting of 1,4-dioxane, 1,3-dioxane, tetrahydrofuran, cyclopentyl methyl ether, tert-butyl-methyl-ether, methanol, and isopropanol.

6. The process according to claim 1, wherein the at least one base is selected from the group consisting of alkoxides, carbonates, bicarbonates, hydroxides, amides, amines, phosphates, and fluorides.

7. The process according to claim 1, wherein the at least one base is selected from the group consisting of sodium tert-butoxide, potassium tert-butoxide, sodium methoxide, sodium amide, lithium diisopropylamide, lithium bis-(trimethyl-silyl)amide, sodium bis-(trimethyl-silyl)amide, triethylamine, trimethylamine, N-dimethylaminopyridine, 1,5-diazabicycl[4.3.0]nonene-5, 5-diazabicycl[5.4.0]undecene-5, lutidine, sodium carbonate, sodium bicarbonate, sodium hydroxide, magnesium carbonate, magnesium bicarbonate, magnesium hydroxide, calcium carbonate, calcium bicarbonate, calcium hydroxide, barium carbonate, barium hydroxide, barium bicarbonate, potassium carbonate, potassium bicarbonate, potassium hydroxide, sodium acetate, potassium acetate, potassium phosphate, calcium acetate, caesium fluoride, potassium hydrogen phosphate, sodium phosphate, sodium hydrogen phosphate, sodium dihydrogen phosphate, potassium dihydrogen phosphate, tributyl amine, pyridine, 1,4-diazabicyclo[2.2. 2]octane, tetramethylguanidine, diisopropyl ethyl amine, sodium trifluoroacetate, potassium trifluoroacetate cesium carbonate, cesium bicarbonate, and cesium hydroxide.

8. The process according to claim 1, wherein the at least one base is sodium tert-butoxide.

9. The process according to claim 1, wherein the at least one metal catalyst complex comprises at least one metal and at least one complexing agent.

10. The process according to claim 9, wherein the at least one metal is selected from the group consisting of nickel, palladium, and platinum.

11. The process according to claim 1, wherein the at least one metal catalyst complex is selected from the group consisting of tetrakis(triphenylphosphine) palladium, dichlorobis(triphenylphosphine) palladium, tris(dibenzylideneacetone) dipalladium [Pd₂(dba)₃], bis(dibenzylideneacetone) dipalladium [Pd(dba)₂], palladium acetate, dichloro(1,5-cyclooctadiene) palladium, and bis[cinnamyl palladium(II)] chloride.

12. The process according to claim 1, wherein the at least one metal catalyst complex is bis[cinnamyl palladium(II)] chloride.

13. The process according to claim 1, wherein the at least one ligand is selected from the group consisting of 5-(di-tert-butylphosphino)-1′,3′,5′-triphenyl-1′H-1,4′-bipyrazole, bis(2-methyl-2-propanyl)(2′,4′,6′-triisopropyl-3,6-dimethoxy-2-biphenylyl)phosphine, dicyclohexyl(2′,4′,6′-triisopropyl-3,6-dimethoxy-[1,1′-biphenyl]-2-yl)phosphine, bis(2-methyl-2-propanyl)(2′,4′,6′-triisopropyl-2-biphenylyl)phosphine, di-(1-adamantyl)-2-morpholinophenylphosphine, tributylphosphine, butyldi-1-adamantylphosphine, (5-diphenylphosphanyl-9,9-dimethylxanthen-4-yl)-diphenylphosphane, (R)-1-[(SP) (diphenylphosphino)ferrocenyl]ethyldicyclohexylphosphine, dicyclohexyl-[2-[2,6-di(propanyloxy)phenyl]phenyl]phosphane, bis[5-(di(1-adamantyl)phosphino)-1′,3′,5′-triphenyl-1′H-[1,4′]bipyrazole, trimethylphosphine, triethylphosphine, tripropylphosphine, triisopropylphosphine, tributylphosphine, tricyclohexylphosphine, trimethylphosphine, triethylphosphite, tripropylphosphite, triisopropylphosphite, tributylphosphite, tricyclohexylphosphite, triphenylphosphine, tri(o-tolyl)phosphine, triisopropylphosphine, tricyclohexylphosphine, 2,2′-bis(diphenylphosphino)-1,1′-binaphthyl (BI NAP), 1,2-bis(dimethylphosphino)ethane, 1,2-bis(diethylphosphino)-ethane, 1,2-bis(dipropylphosphino)ethane, 4,5-bis(diphenylphosphino)-9,9-dimethyl-xanthene (xantphos), 1,1′-bis(diphenylphosphino)ferrocene (dppf), bis(2-(diphenyl-phosphino)phenyl)ether [DPE-phos], 1,2-bis(diisopropylphosphino)ethane, 1,2-bis-(dibutylphosphino)ethane, 1,2-bis(dicyclohexylphosphino)ethane, 1,3-bis(diisopropyl-phosphino)propane, 1,3-bis(dicyclohexylphosphino)propane, 1,4-bis(diisopropyl-phosphino)butane, 1,4-bis(dicyclohexylphosphino)butane, 1,4-bis(diphenylphosphino)-butane (bppb), 2,4-bis(dicyclohexylphosphino)pentane, and 1,1′-bis(diphenylphosphino)ferrocene (dppf).

14. The process according to claim 1, wherein the at least one ligand is 5-(di-tert-butylphosphino)-1′,3′,5′-triphenyl-1′H-1,4′-bipyrazole or bis[5-(di(1-adamantyl)phosphino)-1′,3′,5′-triphenyl-1′H-[1,4′]bipyrazole.

15. The process according to claim 1, wherein
the at least one base is selected from the group consisting of sodium tert-butoxide and potassium tert-butoxide,
the at least one metal complex is bis[cinnamyl palladium (II)] chloride,
the at least one ligand is selected from the group consisting of 5-(di-tert-butylphosphino)-1′,3′,5′-triphenyl-1′H-1,4′-bipyrazole and bis[5-(di(1-adamantyl)phosphino)-1′,3′,5′-triphenyl-1′H-[1,4′]bipyrazole, and
the at least one organic solvent is selected from the group consisting of 1,4-dioxane, 1,3-dioxane, tetrahydrofuran, cyclopentyl methyl ether, tert-butyl-methyl-ether, methanol, and isopropanol.

16. The process according to claim 1, wherein step a) is carried out at a temperature in the range of ≥80° C. to ≤140° C.

17. The process according to claim 1, wherein step a) is carried out at a pressure in the range of ≥1 bar to ≤30 bar.
